(19) United States
(12) Patent Application Publication
(10) Pub. No.: US 2017/0239455 A1
(43) Pub. Date: Aug. 24, 2017

(54) METHODS AND APPARATUS FOR TREATING SITES OF INFECTION

(71) Applicant: BIOCIDE PHARMA, INC., Sudbury, MA (US)

(72) Inventor: James C. Richards, Sudbury, MA (US)

(73) Assignee: I2BIOMED, INC., Concord, MA (US)

(21) Appl. No.: 15/519,186

(22) PCT Filed: Oct. 15, 2015
(86) PCT No.: PCT/US2015/055638

§ 371 Date: Apr. 14, 2017

Related U.S. Application Data
(60) Provisional application No. 62/064,098, filed on Oct. 15, 2014.
(60) Provisional application No. 62/199,505, filed on Jul. 31, 2015.

(51) Int. Cl. A61F 13/40; A61K 9/00; A61K 47/10; A61K 33/18

(52) U.S. Cl. CPC A61M 35/006; A61K 33/18; A61K 9/0014; A61K 47/10

(57) ABSTRACT

Embodiments of the present invention feature apparatus and methods for disinfecting a site of infection with molecular iodine solutions.

### RELATED APPLICATIONS

This application relates to co-pending identically titled provisional application Ser. No. 62/064,098 filed Oct. 15, 2014, and a co-pending identically titled provisional application Ser. No. 62/199,505 filed Jul. 31, 2015, the entire contents of which are incorporated herein by reference.

### STATEMENT REGARDING FEDERAL SPONSORSHIP

Embodiments of the present invention were not conceived or reduced to practice with Federal funds or sponsorship.

### BACKGROUND OF THE INVENTION

The use of iodine compounds for the treatment of topical infections or sites of potential topical infections has been difficult due to the staining properties of most forms of iodine compounds. It would be desirable to have apparatus and methods for applying iodine compounds, as molecular iodine, I₂, as an effective disinfectant for animals and humans. Molecular iodine is an effective microbiocide at concentrations of 100 pfm/ml molecular iodine. Molecular iodine is effective against the virus Ebola as well as other viruses, bacteria and fungi.

### SUMMARY OF THE INVENTION

Embodiments of the present invention are directed to apparatus and methods for applying iodine compounds, as molecular iodine, I₂, as an effective disinfectant for animals and humans. Embodiments of the present invention feature non-staining formulations, which are created at the time of application preserving the molecular non-staining form of iodine.

As used herein, the term “site of a topical infection” refers to places on the skin, or mucosa areas of the oral cavity that have an infection or which potentially may become infected due to a skin or mucosa break, cut, or other injury or merely to disinfect the skin or mucosal surfaces to reduce the possibility of infection or infecting others. For example without limitation one use to reduce the possibility of infection or infecting others is as a hand sanitizer. As used herein, the term “non-staining” means not leaving a discernable colored mark on Caucasian skin from a distance of three feet.

One embodiment of the present invention is directed to an apparatus for administering molecular iodine to a site of a topical infection. The apparatus comprises a first chamber containing molecular iodine in a solvent that does not support the formation of iodine species consisting of iodide ion, triiodine, hypoiodic acid and iodate. The first chamber is opened to release an effective amount of molecular iodine for administration to a site of infection. As used herein. The term “opened” means released by means of removing a seal, tearing open, cutting open, pumping out, or squeezing out of a container an effective amount of molecular iodine.

The solvent is preferably selected from the group consisting of absolute ethanol, absolute isopropanol or absolute methanol. As used herein, the term “absolute” means substantially free of water. The term is used as it is used in a commercial sense to denote that the solution may comprise small limited amounts of water. Water supports the formation of iodine ion, triiodine, hypoiodic acid and iodate through the hydrolysis of molecular iodine.

One embodiment of the present invention further comprises a second chamber. The second chamber contains an application solution for combining with the effective amount of molecular iodine. The application solution has one or more constituents selected from the group consisting of perfumes, surface coating agents, water and ethanol to improve the feel or smell of the effective amount of molecular iodine.

One example of an apparatus with a first chamber is a tear package which is opened at the time of administration. One embodiment features an apparatus further comprising a second chamber. The second chamber contains an application solution for combining with the effective amount of molecular iodine. One example features a second chamber in the form of a tear package which opens upon the release of the effective amount of molecular iodine to form a finished application solution comprising the application solution with an effective amount of molecular iodine. The two solutions are combined immediately prior to administration to the site of the infection. As used herein, the term “immediately” means before a substantial amount of molecular iodine reacts with water to form iodide ion, triiodine, hypoiodic acid and iodate.

A further embodiment of the invention features an apparatus comprising an applicator for applying said finished application solution. The applicator such as a gauze or cotton woven material, swab or plastic applicator may be integrated in the first or second chambers or be held on the surface of the apparatus for removal upon formation of the finished application solution.

A further embodiment of the present invention is directed to a method of treating a site of topical infection. The method comprises the step of administering an effective amount of molecular iodine in a solvent that does not support the formation of iodine species consisting of iodide ion, triiodine, hypoiodic acid and iodate. One embodiment features molecular iodine held in a first chamber in the solvent and said first chamber is opened to release an effective amount of molecular iodine for administration to a site of infection.

The solvent is preferably selected from the group consisting of absolute ethanol, absolute isopropanol and absolute methanol.

One method features a second chamber wherein the second chamber contains an application solution for combining with the effective amount of molecular iodine. The application solution has one or more constituents selected from the group consisting of perfumes, surface coating agents, water and ethanol. As used herein, the term surface coating agent refers to wetting agents, emollients, coloring agents, and surfactants, such as glycerin.

For example, without limitation, where the first chamber is a tear package which is opened at the time of administration the method comprises the step of opening the tear package to release the effective amount of molecular iodine. Wherein the second chamber is also a tear package, the method comprises the step of opening the first chamber and the second chamber to release the effective amount of molecular iodine and form a finished application solution. One embodiment of the method features placing the finished application solution on an applicator for applying to the site of topical infection.

A further embodiment of the present invention is directed to an article of manufacture. The article of manufacture is a formulation for administering an effective amount of molecular iodine to a site of an infection. The formulation comprises molecular iodine in a solvent that does not support the formation of iodine species consisting of iodide ion, triiodine, hypoiodic acid and iodate.

One embodiment features a solvent selected from the group consisting of absolute ethanol, absolute isopropanol and absolute methanol.

One formulation features a mixture of an application solution and molecular iodine in a solvent that does not support the formation of iodine species consisting of iodide ion, triiodine, hypoiodic acid and iodate. The application solution has one or more constituents selected from the group consisting of perfumes, surface coating agents, water and ethanol. The surface coating agent refers to wetting agents, emollients, coloring agents, and surfactants, such as glycerin.

These and other features and advantages will be apparent to those skilled in the art upon viewing the drawings which are described briefly below and upon reading the detailed description that follows.

### BRIEF DESCRIPTION OF THE DRAWINGS

FIG. 1 depicts an apparatus embodying features of the present invention;

FIG. 2 depicts an apparatus embodying features of the present invention;

FIG. 3 depicts an apparatus embodying features of the present invention;

FIG. 4 depicts an apparatus embodying features of the present invention; and,

FIG. 5 depicts an apparatus in cross section embodying features of the present invention.

### DETAILED DESCRIPTION OF THE INVENTION

Embodiments of the present invention will be described in detail as to apparatus and methods for applying iodine compounds, as molecular iodine, I₂, as an effective disinfectant for animals and humans. Embodiments of the present invention feature non-staining formulations, which are created at the time of application preserving the molecular non-staining form of iodine. Those skilled in the arts will readily appreciate embodiments of the present invention can be modified and altered without deviating from the teaching herein. Therefore, the present invention should not be limited to the details presented.

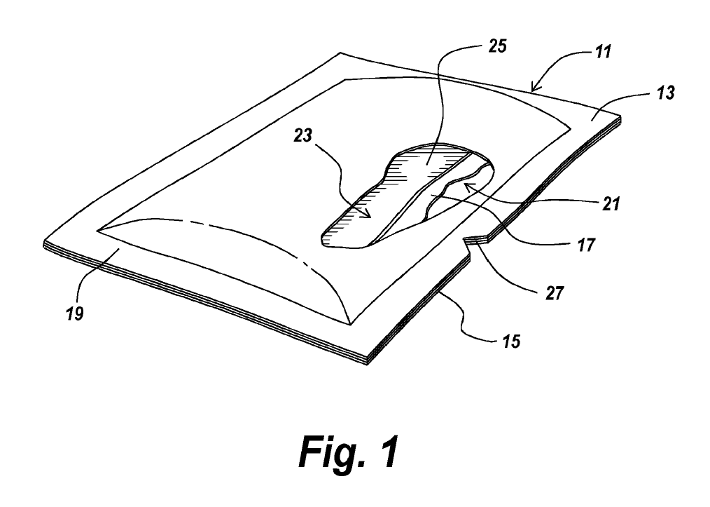

Turning now to FIG. 1, an apparatus for administering molecular iodine to a site of a topical infection, generally designated by the numeral 11, is depicted in partial cutaway. The apparatus 11 comprises a first sheet 13, a second sheet 15 and a third sheet 17. The first sheet 13, second sheet 15 and third sheet 17 have cooperating edge surfaces 19 which allow the first sheet 13, second sheet 15 and third sheet 17 to be affixed, glued, laminated or folded to define a first chamber 21 and a second chamber 23.

First chamber 21 contains molecular iodine in a solvent that does not support the formation of iodine species consisting of iodide ion, triiodine, hypoiodic acid and iodate. The solvent is preferably selected from the group consisting of absolute ethanol, absolute isopropanol or absolute methanol. The molecular iodine is held in the solvent as a saturated solution. First chamber 21 optionally comprises one or more molecular sieve particles [not shown], preferably in a 1-5 angstrom size range, and, more preferably, in about 2-4 angstrom range, or about 3 angstroms, which adsorb free water. The particles may be held in a special receptacle [not shown] secured to the sheets or free in solution. The particles are available from numerous vendors. The molecular iodine solution is stable for periods of six months to one year. That is, at least 90% of the original molecular iodine remains in an active form at the end of the period.

The second chamber 23 holds an applicator 25 which absorbs and holds the molecular iodine in a solvent after the first chamber 21 and second chamber 23 are opened. Applicator 25 facilitates application to the site of topical infection. Those skilled in the art will recognize that the device may readily comprise a single chamber 21 where the applicator 25 is not needed or desired. Where the applicator 25 is substantially inert to the solvent and molecular iodine, the applicator 25 may be contained in the first chamber 21 with the molecular iodine and solvent. As depicted, applicator 25 is a form of wipe or gauze pad known in the art.

The first chamber 21 is opened to release an effective amount of molecular iodine for administration to a site of infection. The term “opened” means released by means of removing a seal, tearing open, cutting open, pumping out, or squeezing out of a first chamber 21 an effective amount of molecular iodine. First sheet 13, second sheet 15 and third sheet 17 have a cooperating tear tab 27 which facilitate the opening of the first chamber 21 and the second chamber 23. After the first chamber 21 and second chamber 23 are opened with respect to each other, the applicator 25 absorbs the molecular iodine and solvent for application to the site of infection.

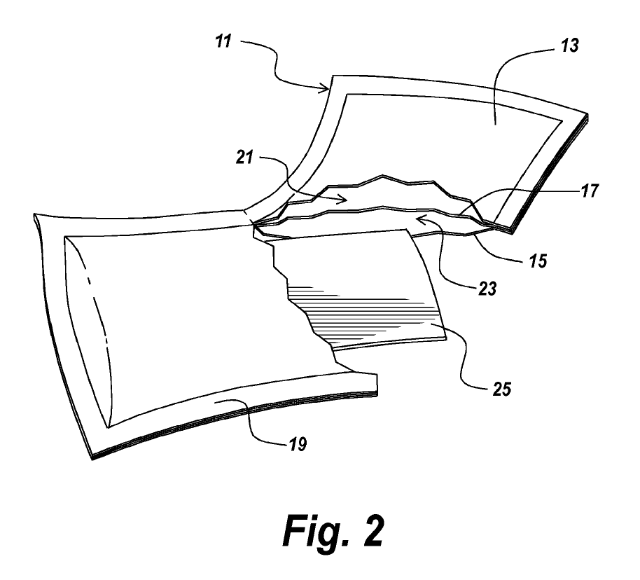

One embodiment features a third sheet 17 which is a breakable seal which upon flexing apparatus 11 by folding or partially folding, breaks and releases the molecular iodine and solvent into the applicator 25. In this embodiment, third sheet 17 is more fragile and breaks or ruptures more readily than the first sheet 13 and second sheet 15. The tear tab 27 is then used to open the first chamber 21 and second chamber 23 as best seen in FIG. 2. The open first chamber 21 and second chamber 23 exposes the applicator 25 to the user which can be removed from the second chamber 23 and applied to the site of the infection. The applicator 25 can be sized to shapes and configurations for a hand or face disinfecting, or for applying and securing to a wound in the nature of an adhesive bandage strip. The form of iodine, molecular iodine is non-staining. The molecular iodine does not persist at the site of application but sublimes into the atmosphere leaving substantially no residue.

One embodiment of the apparatus 11 features a second chamber 23 that contains an application solution for combining with the effective amount of molecular iodine to form an administration formulation. The application solution may be used with or without an applicator 25, and of course, if the apparatus 11 is for use without the applicator 25, it can be omitted from the second chamber 23.

The application solution has one or more constituents selected from the group consisting of perfumes, gelling and surface coating agents, thickening and texture agents, surfactants, preservatives, buffers, water and ethanol to improve the feel or smell of the effective amount of molecular iodine. The presence of preservatives in the applicator solution, in addition to keeping the application solution free of microbial contamination, provides greater persistence to antimicrobial effects of the combined application solution and molecular iodine solution forming the administration formulation. Again, molecular iodine sublimes into the atmosphere over time and is substantially gone in about twenty minutes. Embodiments of the apparatus 11 used without an applicator 25 and with application solutions are well suited as single use hand sanitizers.

One embodiment of a hand sanitizer comprises a molecular iodine solution. The molecular iodine solution is formed with 4.0 mg iodine crystals in 1 ml of absolute ethyl alcohol, or 5.0 mg iodine crystals in 1 ml of absolute ethyl alcohol, or a saturated solution of molecular iodine in absolute ethyl alcohol or a range of 3.0 mg iodine crystals in 1 ml of absolute ethyl alcohol up to a saturated solution. This molecular iodine solution is combined with an application solution to form an administration formulation. One embodiment of the application solution is an aqueous gel.

By way of example, one application solution comprises:

| | |
|---|---|
| Water | 95-98% |
| Gelling Agent (Carbomer) | 0.2-0.6% |
| Texture agent (Propanediol) | 0.1-2.0% |
| Texture agent (Glycerin) | 0.1-0.5% |
| Buffering agent (Aminomethyl Propanol | 0.1-0.5% |
| Preservative | 0.5-2.0% |
| (Benzoic acid) | |
| (Phenoxyethanol) | |
| (Dehydroacetic acid) | |
| (Ethylhexglycerin) | |
| pII 5.0 to 5.5 | |

The application solution and the molecular iodine solution are combined to form an administration formulation in a ratio of 2-6 parts application solution to 1 part molecular iodine solution and, more preferably, four parts application solution to one part molecular iodine solution to form an administration formulation. This administration formulation is effective to kill seven logs of Candida albicans in about 10 seconds and 75 to 99% of Clostridium difficile endospores in one to ten minutes.

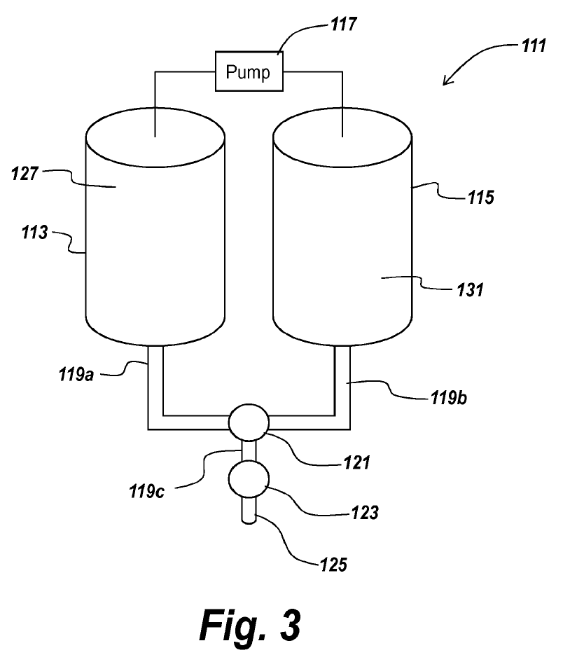

A further embodiment of the present invention is depicted in FIG. 3. An apparatus 111 for dispensing an effective amount of molecular iodine is depicted in a simplified cross-sectional view. The apparatus 111 comprises a first vessel 113, a second vessel 115, pump means 117, conduit means 119a, 119b, and 119c, valve means 121, mixer 123 and outlet 125.

The first vessel 113 defines a first chamber 127 for containing molecular iodine in a solvent that does not support the formation of iodine species consisting of iodide ion, triiodine, hypoiodic acid and iodate. The solvent is preferably selected from the group consisting of absolute ethanol, absolute isopropanol or absolute methanol. The interior walls of first vessel 113 are preferably coated or are of a material inert to molecular iodine and the selected solvent. First vessel 113 optionally comprises one or more molecular sieve particles [not shown], preferably in a 1-5 angstrom size range, and, more preferably, in about 2-4 angstrom range, or about 3 angstroms, which adsorb free water. The number of molecular sieve particles is adjusted for the volume of first vessel 113. For example, a one liter capacity first vessel 113 may have 10 to 1000 particles.

The second vessel 115 defines a second chamber 131 for containing an application solution comprising perfumes, surface coating agents, water, ethanol, thickening and texture materials, surfactants, coloring agents and the like. Although depicted as similar in size to the first vessel 113, the second vessel would normally be sized larger and would release larger quantities of the application solution than the first vessel 113 would release of the molecular iodine solution.

First vessel 113 and second vessel 115 are operably connected with pump means 117. Pump means 117 may take several forms including by way of example without limitation, manual or electric pump apparatus for dispensing liquids, or a source of pressurized fluid for compelling the application solutions and molecular iodine solution from first vessel 113 and second vessel 115. Pressurized systems are well known in the art for dispensing gels and the like. Pump means for pressurized systems may comprise a pressurized can [not shown] in which first vessel 113 and second vessel 115 are sealably placed. In one such embodiment of a pressurized system, first vessel 113 and second vessel 115 are collapsible and shrink in volume as the contents are released under the pressure of the can. Manual and electric pump apparatus comprise piston pumps and bellow systems used as single piston pumps or single bellow or used in tandem to pump and release desired amounts of molecular iodine solution and application solutions.

First vessel 113 and second vessel 115 are in fluid communication through conduit means 119a and 119b. Conduit means 119a and 119b, and conduit means 119c, which will be discussed shortly, may take several forms. By way of example, without limitation, conduit means 119a, 119b and 119c may comprise piping, tubing, devices with molded passages. Conduit means 119a and 119b are in fluid communication with valve means 121. Valve means 121 controls the relative amounts of the molecular iodine solution and applicator solution by means of restrictions, sizing of conduits means 119a and/or 119b, or timing of opening or closing of openings [not shown]. One embodiment features valve means 121 that open and close to the respective vessels to isolate the molecular iodine solution from aqueous components of the application solution that support undesired forms of iodine. For example without limitation, one embodiment features check valves.

Valve means 121 is in fluid communication with mixer 123 via conduit means 119c. Mixer 123 combines the molecular iodine solution and the application solution to distribute the molecular iodine throughout the final dispensed solution, the administration formulation. The mixer 123 may also take several forms, including, by way of example, without limitation, mixing tunnels, membrane infusions and centrifugal mixing chambers. The mixer 123 may be incorporated in the conduits or valve means. The mixer 123 forms the administration formulation and releases the final dispensed solution.

Embodiments of the present invention directed to the method of treating a site of topical infection will now be described with respect to the operation of the apparatus of FIGS. 1-3. Turning now to FIG. 1, an individual desires to treat a site of infection or disinfect a body surface such his or her hands or face, grasps, an apparatus for administering molecular iodine to a site of a topical infection, generally designated by the numeral 11. Apparatus 11 comprises a first sheet 13, a second sheet 15 and a third sheet 17. The first sheet 13, second sheet 15 and third sheet 17 have cooperating edge surfaces 19 which allow the first sheet 13, second sheet 15 and third sheet 17 to be affixed, glued, laminated or folded to define a first chamber 21 and a second chamber 23.

First chamber 21 contains molecular iodine in a solvent that does not support the formation of iodine species consisting of iodide ion, triiodine, hypoiodic acid and iodate. The second chamber 23 holds an applicator 25 which absorbs and holds the molecular iodine in a solvent, after the first chamber 21 and second chamber 23 are opened. Applicator 25 facilitates application to the site of topical infection.

The first chamber 21 is opened to release an effective amount of molecular iodine for administration to a site of infection. For example, where a third sheet 17 has a breakable seal which upon flexing apparatus 11 by folding or partially folding, breaks and releases the molecular iodine and solvent into the applicator 25, the individual folds or bends the apparatus 11. The tear tab 27 is then used to open the first chamber 21 and second chamber 23 as best seen in FIG. 2. The open first chamber 21 and second chamber 23 exposes the applicator 25 to the user which the user removes from the second chamber 23 and applies molecular iodine solution to the site of the infection. The second chamber 23 may also contain an application solution which is mixed with the molecular iodine solution to form an administration formulation during the folding and bending of the apparatus 11.

Turning now to FIG. 3, an individual desires to treat a site of infection or disinfect a body surface such as his or her hands or face, activates a pump means 117 of apparatus 111. Apparatus 111, for dispensing an effective amount of molecular iodine, comprises a first vessel 113, a second vessel 115, pump means 117, conduit means 119a, 119b, and 119c, valve means 121, mixer 123 and outlet 125. Pump means compels a molecular iodine solution from first vessel 113 and an application solution from second vessel 115 through conduit means 119a and 119b to valve means 121.

Valve means 121 controls the relative amounts of molecular iodine solution and application solution which flow through conduit means 119c to mixer 123. Mixer 123 combines the molecular iodine solution and application solution into a more homogeneous final solution which is received by the individual from outlet 125 for application to the hands face or other body surface or site of infection.

Those skilled in the art will recognize devices and apparatus described herewith can be readily used to apply an effective amount of molecular iodine to surfaces as a surface disinfectant. These and other features will be apparent from the following Examples.

### EXAMPLE 1

The first series of experiments were performed to illustrate the preservation and stability of molecular iodine in absolute alcohol. Ethyl alcohol, Absolute, 200 Proof, Reagent ACS was purchased from Science Lab, Houston, Tex. The assay of content indicated a water content of 0.2% in the absolute alcohol. Borosilicate glass vials were heated i.e., 450° F. for one hour to remove water. A standard curve series of molecular iodine in the non-polar solvent heptane (spectroscopy grade) was created by weighing solid crystals of iodine using a Shimadzu Analytical Balance and placing the iodine into duplicate vials as follows: 1 mg, 0.5 mg and 0.1 mg. 10 ml of heptane was added to each vial to create final molecular iodine solutions of 100, 50 and 10 micrograms/ml. The vivid violet color is indicative of molecular iodine in non-polar solvent. The I₂:heptane solution creates a colored solution that represents a quantitative measure of molecular iodine in a non-polar solvent. 100% of the color is due to molecular iodine. Next, the same procedure was repeated in duplicate with solid iodine crystals in heat-treated borosilicate glass vials as before: 1.0 mg, 0.5 mg and 0.1 mg per vial. Next, 10 ml of absolute alcohol was added to each vial. Following complete dissolution at STP 10 ml of heptane was added to each alcohol/iodine solution and mixed on a reciprocal mixer for 30 minutes. All (or >99%) of the available molecular iodine partitioned into the heptane phase of the solvent-solvent-aqueous mixture. The heptane phase in each vial was measured in a Bruckner spectrophotometer at 525 nm. These data indicate that over 50% of the molecular iodine stored in absolute ethanol is maintained as I₂ and a minor amount of staining iodine e.g., I₃— that resulted from the dissociation of I₂ to I— and I₃—.

### EXAMPLE 2

The next series of experiments were designed to demonstrate that molecular iodine in absolute alcohol is a non-staining form of iodine that has antimicrobial properties. The existing literature describes all forms of antimicrobial iodine to be staining. Some examples include tincture of iodine, 10% Betadine, Lugol Solution, and others. Whatman #1 filter paper was employed. Circles were drawn with a #2 pencil. 100 μl (0.1 ml) of each of the indicated solutions were spotted onto the Whatman paper. The spots were allowed to dry for 3-5 min and photographed. The results indicate that iodine maintained in solutions with minimal water content do not form staining solutions. Also, 100 μg/ml and 200 μg/ml in 100% ethanol were spotted on the underside of a forearm and there was no staining immediately following application.

Molecular sieve particles from Sigma-Aldrich were obtained in two forms—3A and 4A. Molecular sieve particles 3A and 4A were pretreated by heating at 450° F. for one hour. This procedure is known to remove residual water. The particles are about 1 mm in diameter and can be counted and added manually. One 3A molecular sieve particle added to 1 ml of absolute alcohol/iodine resulted in the amount of molecular iodine being enhanced/increased 40-50%.

Similarly, 8-10 particles in 10 mL iodine-alcohol, 80-90 particles in 100 mL alcohol-iodine and 150-200 particles in 1000 mL alcohol-iodine produced the same 40-50% increase in molecular iodine. Molecule sieve particles will preserve molecular iodine by capturing unwanted water that might enter the alcohol-iodine chamber thus protecting the molecular iodine from undergoing dissociation.

### EXAMPLE 3

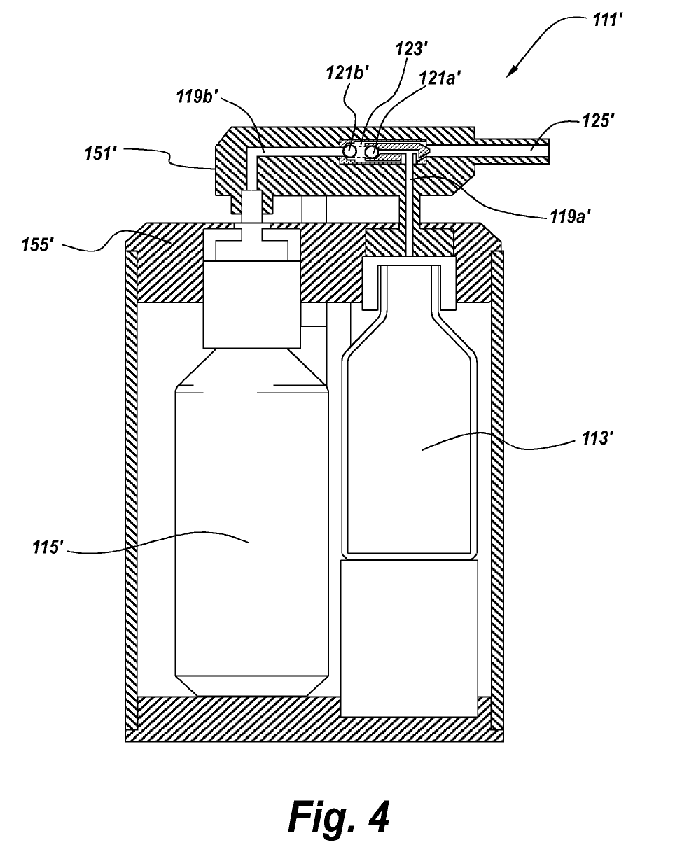

Three application solutions for making three administration formulations were created sequentially over a two month period. The test vehicle was a two chambered delivery device, generally designated by the number 111′, best seen in FIG. 4. The device 111′ has a first vessel 113′ and a second vessel 115′. First vessel 113′ holds a molecular iodine solution as previously described. Second vessel 115′ holds an application solution selected from three application solutions described more fully below.

All three formulations contained identical concentrations of free iodine/molecular iodine or I₂ in 100% ethyl alcohol in first vessel 113′. The free iodine concentration in all three formulations was 4.0 mg/ml in absolute alcohol. In all cases the free iodine concentration was evaluated as described by Pollack and Iny ((U.S. Pat. No. 4,946,673) using extraction of free iodine into a non-polar phase e.g., heptane from the aqueous phase containing the aqueous application solutions 1-3. The cited authors measured the free iodine in the heptane spectrophotometrically at A524 nm since they were determining very low concentrations of I₂ i.e., 2-20 micrograms/ml. This Example used an optical absorbance of A470 nm and/or 524 nm because of significantly higher concentrations of I₂ i.e., 600-4000 micrograms/ml iodine.

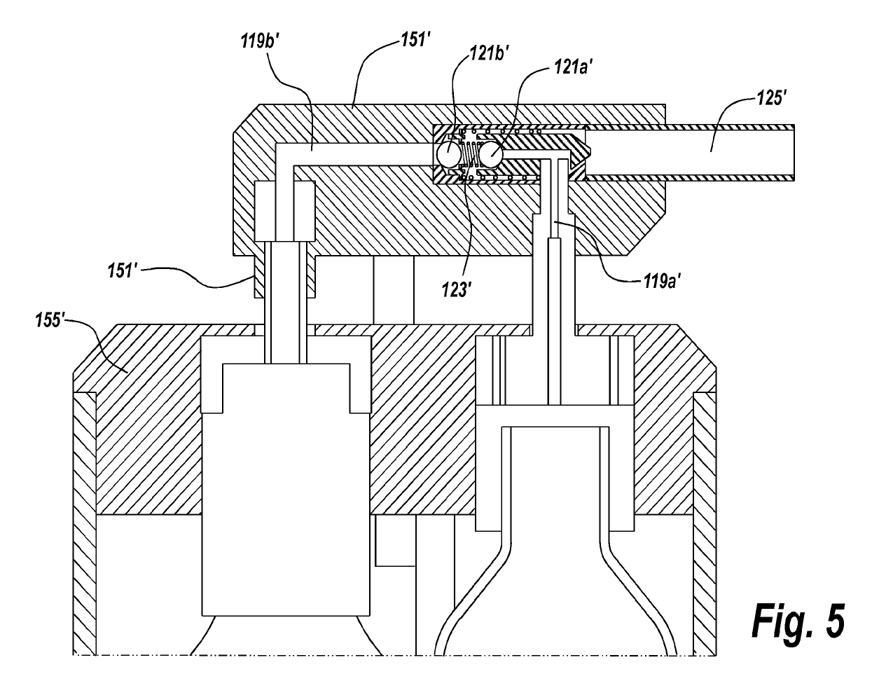

Turning now to FIG. 5, check valves 121a′ and 121b′ prevent entry of aqueous solution held in second vessel 115′ from entering iodine/alcohol in first vessel 113′. The device 111′ has a pump member 151′. Each pumping action achieved by depressing the pump member 151′ with a downward pressure forces 0.62 ml aqueous application solution from second vessel 115′ and 0.18 ml molecular iodine solution from first vessel 113′. The respective solutions are compelled through the check valves 121a′ and 121b′ and into mixer chamber 123′. Mixer chamber 123′ produces a helical mixing action and discharges the combined administration formulation out and onto hands or any surface. The helical mixing action produces a superior combined final solution delivered out onto any solid surface e.g., hands.

In all experiments described below the exact amount of molecular iodine delivered in each mixture was 0.18 ml of 720 micrograms/ml iodine/alcohol. The actual concentration of molecular iodine delivered with each pump is therefore 720 micrograms iodine/alcohol. Similarly, the exact amount of aqueous application solution 1, 2 or 3 delivered with each pump action is 0.62 ml. The final volume of hand sanitizer, administration formulation, a delivered onto hands is 0.9 ml/pump. Therefore, the only experimental variable in each evaluation of Formula 1 vs Formula 2 vs Formula 3 is the chemical composition of each aqueous formula contained in second vessel 115′.

Aqueous Formula 1

| INCI Name | % by weight | Manufacturer |
|---|---|---|
| Water | 97.0 | |
| Carbomer | 0.4 | Lubrazol |
| Propanediol | 1.0 | DuPont |
| Glycerin | 0.5 | Sigma |
| PEG-50 Shea Buffer | 0.5 | Vantage |
| Preservative: | 0.6 | Shulke |
| Benzoic acid, Phenoxyethanol | | |
| Dehydroacetic acid, ethyhexyglycerin | | |
| Poryaminopropyl biguanidine | | |

Aqueous Formula 2

| INCI Name | % by weight | Manufacturer |
|---|---|---|
| Water | 97.2 | |
| Carbomer | 0.2 | Lubrazol |
| Propanediol | 1.0 | DuPont |
| Glycerin | 0.5 | Sigma |
| PEG-50 Shea Buffer | 0.5 | Vantage |
| Preservative: | 0.6 | Shulke |
| Benzoic acid, Phenoxyethanol | | |
| Dehydroacetic acid, ethyhexyglycerin | | |
| Polyaminopropyl biguanidine | | |

Aqueous Formula 3

| INCI Name | % by weight | Manufacturer |
|---|---|---|
| Water | 96.8 | |
| Carbomer | 0.4 | Lubrazol |
| Propanediol | 1.0 | DuPont |
| PEG-50 Shea Buffer | 0.5 | Vantage |
| Aminomethyl Propanol | 0.3 | |
| Preservative: | 1.0 | |
| Benzoic acid, Phenoxyethanol | | |
| Dehydroacetic acid, Ethyhexyglycerin | | |

Experiment 1. Evaluation of Formula 1.

Purpose: To determine the antimicrobial efficacy of delivery device 111′ loaded with Formula 1 and iodine/alcohol as described above. The delivery device 111′ containing Formula 1 was sent to as CRO in Texas (Antimicrobial Test Laboratories/ATL) and evaluated for microbial killing/efficacy and percentage kill at various time points against the following pathogens:

| | |
|---|---|
| Staphylococcus aureus(MRSA) | 99.99999% (<10 sec) |
| E. coli | 99.99999% (<10 sec) |
| Candida albicans (yeast/fungi) | 99.99999% (<10 sec) |
| Hepatitis A (RNA virus) | 99.99% (30 sec) |
| Norovirus (“cruise ship virus”) | 99.99% (30 sec) |
| Influenza A virus “flu virus”) | 99% (30 sec) |
| Clostridium difficile spores (C. dif″) | 50% killed in one hour |

Experiment 2. Evaluation of Formula 2.

Purpose: To determine the antimicrobial efficacy of delivery device 111′ loaded with Formula 2 and iodine/alcohol as described above. The delivery device 111′ containing Formula 2 was sent to as CRO in Texas (Antimicrobial Test Laboratories/ATL) and evaluated for microbial killing/efficacy against the following pathogens:

| | |
|---|---|
| Staphylococcus aureus(MRSA) | 99.99999% (<10 sec) |
| E. coli | 99.99999% (<10 sec) |
| Candida albicans (yeast/fungi) | 99.99999% (<10 sec) |
| Hepatitis A (RNA virus) | 99.99% (30 sec) |
| Norovirus (“cruise ship virus”) | 99.99% (30 sec) |
| Influenza A virus “flu virus”) | 99% (30 sec) |
| Clostridium difficile spores (C. dif″) | 90% killed in 30 min) |

Results:

Experiments 1 and 2 gave very similar results with a slight improvement in the killing of Clostridium difficile endospores, a very resistant pathogen. It is surprising and unexpected that there was any improvement based on simply lowering the carbomer concentration 50%. Formula 3 was sent to ATL for testing antimicrobial killing. Only the vegetative bacteria and yeast and c. difficile were tested. C. difficile endospores represented the most significant challenge for antimicrobial killing efficacy. The results were surprising as shown below.

Experiment 3. Evaluation of Formula 3.

Purpose: To determine the antimicrobial efficacy of delivery device 111′ loaded with Formula 3 and iodine/alcohol as described above. The delivery device 111′ containing Formula 3 was sent to as CRO in Texas (Antimicrobial Test Laboratories/ATL) and evaluated for microbial killing/efficacy against the following pathogens:

| | |
|---|---|
| Staphylococcus aureus(MRSA) | 99.99999% |
| E. coli | 99.99999% |
| Candida albicans (yeast/fungi) | 99.99999% |
| Clostridium difficile spores (C. dif″) | 99.99% killed under 1 min; |
| | 99.999% killed under 3 min; |
| | 99.999% killed under 5 min |
| Under 10 sec | |

The Formula 3 was significantly more efficient in killing C. difficile endospores and this result was surprising considering no known hand sanitizer can kill C. difficile endo spores and in fact approaches the definition of a sterilant based on US FDA and EPA definitions.

The results were and are surprising and may lead to a revolutionary hand sanitizer capable of kill spores on human skin and possibly reducing the spread of C. difficile in healthcare settings.

Thus, the invention has been described in detail as an apparatus and method for treating a site of infection with the understanding that the present invention is subject to modification and alteration and should not be limited to the precise details set forth herein but should encompass the subject matter of the claims that follow and their equivalents.

1. An apparatus for administering molecular iodine to a site of a topical infection comprising:
a. a first chamber containing molecular iodine in a solvent that does not support the formation of iodine species consisting of iodide ion, triiodate, hypoiodic acid and iodate wherein said first chamber is opened to release an effective amount of molecular iodine for administration to a site of infection.

2. The apparatus of claim 1 wherein said solvent is absolute ethanol.

3. The apparatus of claim 1 further comprising an applicator.

4. The apparatus of claim 1 wherein said apparatus further comprises a second chamber, said second chamber containing an application solution for combining with said effective amount of molecular iodine.

5. The apparatus of claim 4 wherein said application solution has one or more constituents selected from the group consisting of perfumes, surface coating agents, water and ethanol.

6. The apparatus of claim 1 wherein said first chamber is a tear package which is opened at the time of administration.

7. The apparatus of claim 6 wherein said apparatus further comprises a second chamber, said second chamber containing an application solution for combining with said effective amount of molecular iodine.

8. The apparatus of claim 7 wherein said second chamber is a tear package which opens upon the release of said effective amount of molecular iodine to form a finished application solution comprising said application solution with an effective amount of molecular iodine.

9. The apparatus of claim 8 further comprising an applicator for applying said finished application solution.

10. The apparatus of claim 1 wherein the molecular iodine in a solvent that does not support the formation of iodine species consisting of iodide ion, triiodate, hypoiodic acid and iodate is non-staining to human skin.

11. A method of treating a site of topical infection comprising the steps of administering an effective amount of molecular iodine in a solvent that does not support the formation of iodine species consisting of iodide ion, triiodate, hypoiodic acid and iodate.

12. The method of claim 11 wherein said molecular iodine is held in a first chamber in said solvent and said first chamber is opened to release an effective amount of molecular iodine for administration to a site of infection.

13. The method of claim 11 wherein said solvent is absolute ethanol.

14. The method of claim 12 wherein said apparatus further comprises a second chamber, said second chamber containing an application solution for combining with said effective amount of molecular iodine.

15. The method of claim 14 wherein said application solution has one or more constituents selected from the group consisting of perfumes, surface coating agents, water and ethanol.

16. The method of claim 12 wherein said first chamber is a tear package which is opened at the time of administration and said method comprises the step of opening the tear package to release said effective amount of molecular iodine.

17. The method of claim 12 wherein an application solution is held in a second chamber, said second chamber containing an application solution for combining with said effective amount of molecular iodine, said method comprising the step of opening said first chamber and said second chamber to release said effective amount of molecular iodine and form a finished application solution.

18. The method of claim 17 wherein said second chamber is a tear package which opens upon the release of said effective amount of molecular iodine to form a finished application solution comprising said application solution with an effective amount of molecular iodine.

19. The method of claim 18 wherein said effective amount of molecular iodine is placed on an applicator for applying said finished application solution.

20. As an article of manufacture, a formulation for administering an effective amount of molecular iodine to a site of an infection comprising molecular iodine in a solvent that does not support the formation of iodine species consisting of iodide ion, triiodate, hypoiodic acid and iodate.

21. The article of manufacture of claim 20 wherein said solvent is selected from the group consisting of absolute ethanol, absolute isopropanol.

22. The article of manufacture of claim 21 further comprising an applicator solution, said applicator solution having one or more constituents selected from the group consisting of perfumes, surface coating agents, water and ethanol, which applicator solution is combined with the molecular iodine in a solvent that does not support the formation of iodine species consisting of iodide ion, triiodate, hypoiodic acid and iodate immediately prior to application to a site of infection.
